(19) United States
(12) Plant Patent Application Publication
(10) Pub. No.: US 2013/0276188 P1
(43) Pub. Date: Oct. 17, 2013

(54) Azalea plant named 'MNIHAR021'

(50) ;

(71) Applicant: Ella Ruth Harris, (US)

(72) Inventor: James O. Harris, Lawrenceville, GA (US)

(21) Appl. No.: 13/506,319

(22) Filed: Apr. 11, 2012

(51) Int. Cl. A01H 5/00

(52) U.S. Cl. PLT/240

(57) ABSTRACT

A new and distinct variety of azalea plant with spring and fall blooming, attractive, showy, single dark pink flowers, easily propagated by semi-hardwood cuttings in late spring through summer, moderate growth rate under normal fertilization and moisture conditions; broadly globose and freely branching in habit, grows well in containers, thrives in shade or sun and is hardy in Zone 6, is disclosed.

### GENUS AND SPECIES

Rhododendron sp.

### VARIETY DENOMINATION

‘MNIHAR021’

### BACKGROUND OF THE NEW PLANT

The present invention is a new and distinct variety of evergreen azalea of the genus Rhododendron. This new azalea, hereinafter referred to as ‘MNIHAR021’, originated from a planned cross hybridization between the female azalea plant ‘Martha Hitchcock’ (unpatented) and the male azalea plant code named ‘N1’ (unpatented) in a controlled environment in Lawrenceville, Ga. in 2004. The present invention has a blooming period in mid March to early April and early September until frost. ‘MNIHAR021’ has a compact, broadly globose, freely branching growth habit, and cold hardiness.

The new plant was first propagated via semi-hardwood cuttings in 2005 in Dearing, Ga. and has been asexually reproduced repeatedly by semi-hardwood cuttings in Dearing, Ga. for over 6 years and four generations. ‘MNIHAR021’ has been found to retain its distinctive characteristics through successive asexual propagations via semi-hardwood cuttings.

Plant Breeder's Rights for this variety have not been applied for. ‘MNIHAR021’ has not been made publicly available or sold more than one year prior to the filing date of this application.

### SUMMARY OF THE INVENTION

The following are the most outstanding and distinguishing characteristics of this new cultivar when grown under normal horticultural practices in Dearing, Ga.

1. Spring and fall blooming;

2. Attractive, showy, dark pink colored flowers;

3. Numerous single flowers;

4. Easily propagated by semi-hardwood cuttings in late spring through summer;

5. Moderate growth rate under normal fertilization and moisture conditions;

6. Compact, broadly globose, freely branching growth habit;

7. Grows well in containers;

8. Thrives in shade or sun; and

9. Hardy in Zone 6.

### DESCRIPTION OF THE PHOTOGRAPHS

This new azalea variety is illustrated by the accompanying photographs. The colors shown are as true as can be reasonably obtained by conventional photographic means. The photographs are of 7 year-old plants grown in filtered shade and full sun in 2011 in Dearing, Ga.

FIG. 1 is a close-up showing mature foliage.

FIG. 2 shows growth habit and spring flowering of a plant.

FIG. 3 is a close-up showing flower buds and immature foliage.

FIG. 4 is a close-up showing flower size, form, and color.

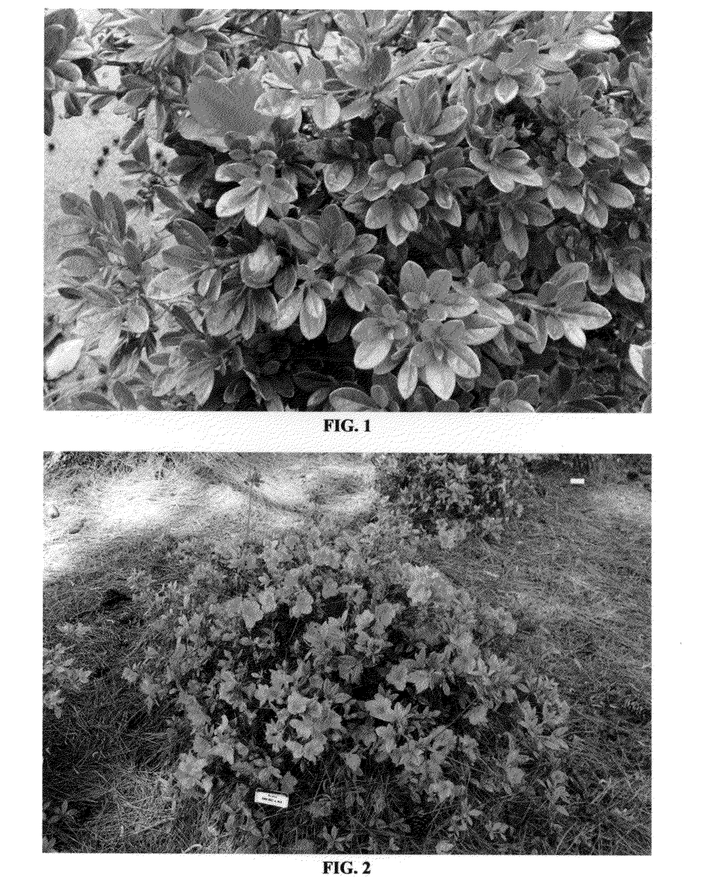

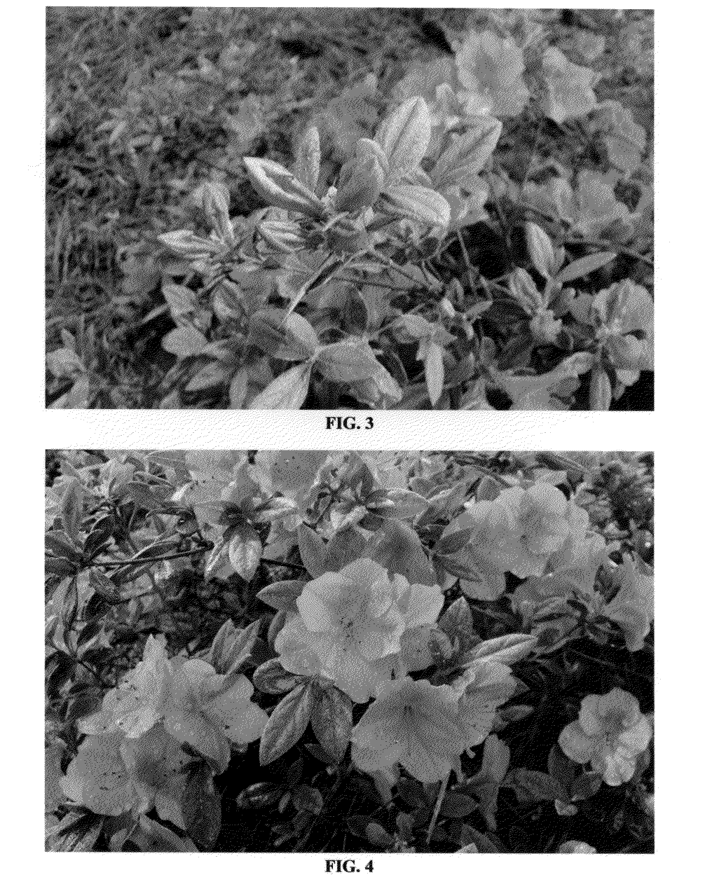

### DETAILED BOTANICAL DESCRIPTION OF THE NEW PLANT

The following is a detailed description of the new variety of azalea based on observations made of three and four-year-old plants grown in trade and three-gallon containers according to wholesale commercial production conditions, under full-sun and in an evaluation bed under semi-shade conditions in Dearing, Ga. in the spring, summer, and fall seasons of 2008, 2009, 2010, and 2011. The colors of the various plant parts are detailed with reference to The Royal Horticultural Society Colour Chart (2001).
Classification:
   Family.—Ericaceae.
   Species.—Rhododendron sp.
   Common name.—Azalea.
   Commercial name.—‘MNIHAR021’.
Parentage:
   Female parent.—The azalea plant ‘Martha Hitchcock’ (unpatented).
   Male parent.—The azalea plant code named ‘Ni’ (unpatented).
Plant:
   Form.—Compact, broadly globose.
   Texture.—Medium.
   Height.—60.96 cm to 91.44 cm.
   Width.—91.44 cm to 121.92 cm.
   Growth habit.—Compact, broadly globose, freely branching, dense in nature.
   Growth rate.—Moderate growth rate under normal fertilization and moisture conditions. In a period of 4 years from a rooted cutting the plant reaches a height of 28.0 cm and a spread of 64.0 cm. The growth rate is normally averages about 6.6 cm per year; the plant reaches a height of 60.96 cm to 91.44 cm at maturity while maintaining a compact habit due to the abundant deliquescent branch development.
   Spring growth.—The date of initial spring growth is March 26, followed by continuous growth through fall.
   Life cycle.—Perennial and evergreen.
Leaves:
   Arrangement.—Alternate, simple, pubescent, and evergreen.
   Shape.—Oblanceolate to obelliptic.
   Apex.—Mucronate.
   Base.—Attenuate.
   Margin.—Entire.
   Length.—3.1 cm.
   Width.—1.4 cm.
   Venation pattern.—Pinnate; the mid-veins and laterals are impressed on the upper surface and the prominent on the lower surface.
   Immature leaf upper surface.—Color: Semi-glossy, RHS 143A (yellow-green) Pubescence: Strigose Pubescence color: RHS N155A (white).
   Immature leaf lower surface.—Color: Matte to semi-glossy, RHS 144A (yellow-green) Pubescence: Strigose with some scabrous concentrated along the veins Pubescence color: RHS N155A (white).
   Mature leaf upper surface.—Color: Semi-glossy to matte, RHS 139A (green) Pubescence: Moderate strigose Pubescence color: RHS 165C (greyed-orange).
   Mature leaf lower surface.—Color: Semi-glossy to Matte, RHS 147B (yellow-green) Pubescence: Strigose with some scabrous Pubescence color: RHS N155B (white).
   Petiole.—Length: 0.5 cm Diameter: 0.1 cm Pubescence: Strigose Length: 0.1 cm Color: Immature: RHS N155A (white) Mature: RHS 165C (greyed-orange) Color: Immature: RHS 145A (yellow-green) Mature: RHS 144B (yellow-green).
Stems:
   Form.—Branched at terminal buds with some from auxiliary buds at leaf nodes.
   Young stems (actively growing, less than one year old).—Color: RHS 144A (yellow-green) Pubescence: Strigose Pubescence color: Begins RHS N155A (white) maturing to RHS 165C (greyed-orange) Length of fully elongated flush: 4.0 cm on average Diameter: 0.2 cm.
   Mature stems (one year old and older).—Color: RHS 199A (grey-brown) with areas of RHS 165A (greyed-orange) Pubescence: Strigose, the quantity decreases with age of the stem Pubescence color: RHS 199C (grey-brown) in color Length: 10.0 cm on average for a full year's growth Diameter: 0.3 cm in second year Pith: Solid and uniform Internode length: Average 1.0 cm on actively growing vegetative stems.
Flower buds:
   Arrangement and form.—One to three buds at terminal; with each flower borne in groups of one to three per bud, sheathed by one to three modified leaf bracts.
   Length.—Average 1.2 cm.
   Width.—0.4 cm.
   Color.—RHS 139A (green).
   Shape.—Ovate.
   Apex.—Acute.
   Base.—Rounded.
   Length (at tight bud).—1 cm.
   Diameter (at tight bud).—0.6 cm.
   Color (of scales).—Immature: RHS 145A (yellow-green) Mature: RHS 144B. (yellow-green) with areas of RHS 47D (red).
   Texture.—Matte; sericeous with some lanulose pubescence.
   Pubescence color.—RHS N155A (white) maturing to RHS 164A (greyed-orange).
   Pedicel.—Length: 0.7 cm at point bud begins to open Diameter: 0.2 cm Texture: Pubescent, Sericeous pubescence Pubescence color: RHS N155A (white) Color: RHS 144A (yellow-green).
Calyx:
   Calyx diameter.—1.1 cm.
   Sepal length.—0.5 cm.
   Sepal width.—0.2 cm.
   Sepal quantity.—5 per calyx.
   Diameter.—1.0 cm, cup shaped.
   Texture.—Pubescent, Sericeous with some tomentose pubescence, most near the margin on the lower side. Pubescence color: RHS N155A (white)
   Color.—RHS 144A (yellow-green).
Flowers:
   Type.—Perfect, single flowers.
   Shape.—Open funnel-shaped.
   Flowering habit and period.—Borne on the current season's growth; Three weeks beginning in mid March in Dearing, Ga.; flowering resumes early September and continues until frost which can be as late as November or December.
   Lastingness of flowers on the plant.—4 to 6 days in the sun; 5 to 8 days in the shade.
   Fragrance.—Absent.
   Diameter.—5.5 cm.
   Depth.—3.0 cm.
   Petals.—Quantity per flower and arrangement: 5 imbricate petals that are fused at the base and remain fused up to 1.0 cm from the base Shape: Obovate Apex: Rounded with some retuse Base: Fused Margin: Entire, undulate Texture (both surfaces): Glabrous Length (of petals without spots): 3.7 cm Width (of petals without spots): 2.6 cm Color: Upper surface: RHS 68B (red-purple) the upper surface of the spots are RHS 71A (red-purple) Lower surface: RHS 67B (red-purple), lower surface of spots are not visible.
Reproductive parts:
   Pistil.—Quantity and form: Single, non-petaloid Length: 3.2 cm Diameter: 0.15 cm.
   Stigma.—Color: RHS 60A (red-purple) Diameter: 0.1 cm.
   Style color.—RHS 58B (red-purple) and RHS 63B (red purple) at the apex.
   Ovary.—Pubescent, sericeous with 5 locules.
   Ovary color.—RHS 141A (green) with RHS N155A (white) pubescence.
   Stamens.—Quantity: 5 per flower; non-petaloid Filament length: 2.5 cm Filament width: 0.05 cm Color: RHS 63C (red-purple).
   Anther.—Length: 0.2 cm Width: 0.1 cm Color: RHS N77A (purple) Pollen: Abundant Color: RHS 158B (yellow-white).
Fruit and seed: Observed
   Maturity.—The capsule matures in about 6 months in Dearing, Ga. and the fruit set is moderate and contains about 100 to 150 non-winged seeds.
   Capsule length.—0.8 cm.
   Capsule width.—0.6 cm.
   Capsule color.—RHS 146B (yellow-green).

### CULTURE

‘MNIHAR021’ grows well in a wide range of conditions and tolerates sun to shade. ‘MNIHAR021’ prefers moist, well-drained soil that is rich in organic matter and responds well to mulching and medium applications of fertilizer. ‘MNIHAR021’ does best in soil with a pH of 5.0 to 5.5 and is propagated with semi-hardwood cuttings in late spring through the summer. Azaleas root in five to six weeks with a high percentage of rooting (80% plus). Tissue culture is also an effective means of propagation for azaleas.

### DISEASE AND INSECTS

Susceptible to lace bugs, root weevils, and spider mites. Fungal and bacterial pathogens have not been observed, but no resistance testing has been performed.

### COMPARISON WITH PARENTAL AND COMMERCIAL VARITIES

In Table 1, ‘MNIHAR021’ is compared to parental varieties ‘Martha Hitchcock’ (unpatented) and ‘N1’ (unpatented).

TABLE 1

| Characteristic | ‘MNIHAR021’ | ‘Martha Hitchcock’ | ‘N1’ |
|---|---|---|---|
| Plant Height | 60.96 cm to 91.44 cm | 91.44 cm | N/A |
| (Mature) | | | |
| Flower Diameter | 5.5 cm | 7.62 cm | 8.89 cm |
| Flower Form | Single | Single | Single |
| Flower Color | Dark pink | White with purple | Red |
| | | margin | |
| Bloom Period | Mid March to early | April | Spring and |
| | April and early | | Fall |
| | September until frost | | |
| Hardy Zone | 6 | 6 | N/A |
| Stamen Number | 5 | 5 | N/A |
| Stamen Type | Non-petaloid | Non-petaloid | Non- |
| | | | petaloid |

When ‘MNIHAR021’ is compared to the commercial variety ‘Roblen’ (U.S. Plant Pat. No. 16,248), ‘MNIHAR021’ has a compact, broadly globose growth habit with a height of 53.0 cm and a spread of 94.0 cm in eight years, while ‘Roblen’ has a height of 91.44 cm and a spread of 60.96 cm in six years. Additionally, ‘MNIHAR021’ produces dark pink flowers, while ‘Roblen’ has bright red flowers.

I claim:

1. A new variety of azalea plant named ‘MNIHAR021’ as herein shown and described.
